(19) United States
(12) Patent Application Publication
(10) Pub. No.: US 2014/0154728 A1
(43) Pub. Date: Jun. 5, 2014

(54) COMPOSITIONS, SYSTEMS AND METHODS BASED ON THE DIFFERENTIATION OF EPICARDIAL CELLS TO TYPICAL AND ATYPICAL FATES

(71) Applicant: UNIVERSITY OF SOUTHERN CALIFORNIA, Los Angeles, CA (US)

(72) Inventors: Henry M. Sucov, Manhattan Beach, CA (US); Yukiko YAMAGUCHI, Los Angeles, CA (US)

(73) Assignee: UNIVERSITY OF SOUTHERN CALIFORNIA, Los Angeles, CA (US)

(21) Appl. No.: 14/095,993

(22) Filed: Dec. 3, 2013

Related U.S. Application Data
(60) Provisional application No. 61/732,881, filed on Dec. 3, 2012.

(51) Int. Cl. G01N 33/50

(52) U.S. Cl. CPC G01N 33/5044
USPC 435/29; 435/325

(57) ABSTRACT

Disclosed are epicardium-derived mesenchymal cells (EPDCs) that differentiate to fates that are commonly adopted by mesenchymal cells elsewhere in the body, but not currently associated with the epicardium. Also disclosed are methods, systems and assays relating to adipocyte and/or osteogenic differentiation in EPDCs that are often related to disease or disregulated states, i.e. the “atypical” fates of epicardium-derived cells. Disclosed are specific EPDC cells that model both the typical fates and atypical fates of EPDCs, and resultant uses of such EPDC cells in systems, methods, compositions and drug discovery.

### CROSS-REFERENCE TO RELATED APPLICATIONS

This application claims the benefit of U.S. Provisional Application No. 61/732,881, filed Dec. 3, 2012, which is incorporated herein by reference in its entirety.

### STATEMENT REGARDING FEDERALLY SPONSORED RESEARCH

This invention was made with government support under Grant No. HL070123 awarded by the National Institute of Health. The government has certain rights in the invention.

### BACKGROUND

During heart development and in the adult heart following injury, the epicardium undergoes transformation to generate mesenchymal cells. The two established fates of epicardium-derived mesenchymal cells (EPDCs) are to become smooth muscle and fibroblasts. However, there has been relatively little attention directed to the ability or potential of the epicardium to differentiate to fates that are commonly adopted by mesenchymal cells elsewhere in the body, but not currently associated with the epicardium. Adipocyte and osteogenic differentiation are two such “atypical” fates. Both atypical differentiation pathways are in fact followed naturally and in contexts that are clinically relevant, and yet have received little attention in past studies. As such, there is a need for compositions, methods and systems which can effectively model adipocyte and osteogenic differentiation in EPDCs.

Coronary calcification is characterized by the presence of mineralized calcium in the walls of atherosclerotic coronary vessels. Not all atherosclerotic plaque is calcified, but the presence of calcification is recognized as a major risk factor for plaque rupture [3-5], which can be followed by rapid vessel occlusion and myocardial infarction. Osteoblast-like cells are responsible for mineral deposition in coronary calcification. However, there has been a lack of a suitable experimental model system for coronary calcification and of assays and systems that can identify potential drug candidate that selectively inhibit coronary calcification.

### BRIEF SUMMARY

One aspect of the present invention is directed to the ability and/or potential of EPDCs to differentiate to fates that are commonly adopted by mesenchymal cells elsewhere in the body, but not currently associated with the epicardium. Specifically, one aspect of this invention is directed to methods, systems and assays relating to adipocyte and/or osteogenic differentiation that are often related to disease or disregulated states, i.e. the “atypical” fates of epicardium-derived cells—as wells as the “typical fates”. Without being limited to theory, it is believed that both allegedly “atypical” differentiation pathways are in fact followed naturally in the body and in contexts that are clinically relevant, and yet have received little attention in the past. One aspect of the present invention is directed to specific EPDC cells that model both the typical fates and atypical fates of EPDCs, and resultant uses of such EPDC cells in systems, methods and compositions.

Another aspect of the present invention is the based on the finding that mouse epicardium has the capacity to generate osteoblasts and adipocytes, although it normally doesn't do so in vivo and so mouse EPDCs as described hereincan form the basis for research tools, assays, methods and systems for the elucidation of osteoblast-like and adipocyte fates of epicardial derived cells as well as smooth muscle cells or fibroblasts.

A method of identifying a compound for preventing and/or treating coronary calcification or a disease state associated with coronary calcification comprises (1) obtaining a EPDC cell, preferably an MEC1 cell, and providing the MEC1 cell(s) in a medium comprising a phosphate source, (2) adding a test compound to the medium, in the presence of the EPDC cells and the phosphate source, (3) measuring a calcium mineralization response, (4) comparing the mineralization response in the sample comprising the EPDC cells, phosphate source and test compound with a calcium mineralization response of in a control without the test compound, and (5) selecting a test compound that reduces the mineralization response in the presence of test compound compared to a mineralization response in the control. If the test compound is found to have reduced the calcium mineralization compared to calcium mineralization in a medium having EPDC cells in which the test compound is not administered, the test compound can be identified as a compound for preventing and/or treating coronary calcification or a disease state associated with coronary calcification. Although the method has been described in terms of adding a test compound to a medium comprising EPDC and the phosphate source, the methods of the present invention may also be practice by adding EPDC and a phosphate source to a medium comprising a test compound as would be understood by those ordinarily skilled.

The measured calcium mineralization response is preferably, but is not limited to, the deposition of calcium mineralized material deposited in an extracellular matrix. In such embodiments, the EPDC cells of the invention should generally be at a high enough density in the medium and the phosphate source in the medium should present at a concentration sufficient to produce a calcium mineralized material dispersed or deposited in an extracellular matrix, and preferably a calcium phosphate mineralized material dispersed or deposited in the extracellular matrix. The calcium mineralized material may be detected and quantified by any suitable method known to those ordinarily skill in the art, including visual observation or staining.

The composition used in connection with the methods generally comprise: EPDC cells of the present invention, preferably MEC1 cells, in a medium, preferably an aqueous medium, and at least one phosphate source. The EPDC cells of the invention are generally at a high enough density in the medium and the phosphate source in the medium is present at concentrations sufficient to produce a calcium mineralized material dispersed or deposited in an extracellular matrix, and preferably a calcium phosphate mineralized material dispersed or deposited in the extracellular matrix. The calcium source is not particularly limited and may generally be any calcium source known to those ordinarily skilled to be useable in connection with the deposition of calcium mineralization from osteoblasts. In an especially preferred embodiment, the calcium source is beta-glycerolphosphate. Preferably, the medium further includes ascorbic acid, and preferably the medium includes both beta-glycerolphosphate and ascorbic acid (bGP+AA).

### BRIEF DESCRIPTION OF THE FIGURES

The file of this patent contains at least one drawing executed in color. Copies of this patent with color drawings will be provided by the Office upon request and payment of the necessary fee

FIG. 1A shows MEC1 cell osteogenic differentiation visualized by alizarin staining at various days uninduced or after bGP+AA treatment. FIG. 1B shows osteogenic differentiation in MEC1 and primary mouse EPDC cultures after 14 days without or with bGP+AA differentiation induction, stained with alizarin. FIG. 1C shows 3T3LI cells exhibit no osteogenic differentiation visualized by alizarin staining at various days uninduced or after bGP+AA treatment.

FIG. 2. Runx2 and PPARγ gene expression in mouse and rabbit. cDNA samples were assayed by PCR using species-appropriate primers. Reference samples are adult peripheral body fat and forelimb (including bone; presumably also including fat within bone marrow and dermis). Primary: primary EPDC cultures from adult hearts. Note the absence of PPARγ expression in primary mouse and MEC1 cells and prominent PPARγ expression in rabbit primary EPDCs (arrows).

FIG. 3. Runx2 expression by ISH at E13.5. Sections were hybridized with a sense strand control or with an antisense Runx2 probe. Note scattered positive cells (examples indicated by solid arrows in the interventricular septum and right ventricle wall), and surrounding the forming rib bone. Note also light epicardial expression (open arrows). Epifluorescence in red blood cells (RBC) with the sense probe is not real signal.

FIG. 4. Adipogenic differentiation. MEC1 or primary mouse or pig EPDC cultures were uninduced or treated with DII and/or the PPARγ ligand rosiglitazone. Adipogenic differentiation was visualized by the presence of neutral lipid via oil red 0 staining MEC1 cells were first infected with adenovirus expressing GFP or the indicated factors. Note the presence of abundant adipocytes when pig cells or MEC1 cells expressing PPARγ or C/EBPβ were induced to differentiate. Rabbit cells were like pig cells in competence to undergo adipogenesis (not shown). Pig and rabbit cells were sometimes passaged once before experiments to eliminate any previously differentiated adipocytes, although this step was not necessary.

FIG. 5. A simplified scheme of regulation of adipogenic differentiation.

FIG. 6. Coronary calcification in mice. Trans-diaphragmatic cryoinjury was administered to adult mice with normal kidney function (top) or with 5/6 nephrectomy (bottom), and sections then stained with alizarin. The injury site (asterisk) is in the lower RV close to the apex and ventricular septum. In mice with nephrectomy+cryoinjury, small vessels with alizarin-staining deposits were readily observed (arrows). These were not seen with cryoinjury alone or with nephrectomy alone.

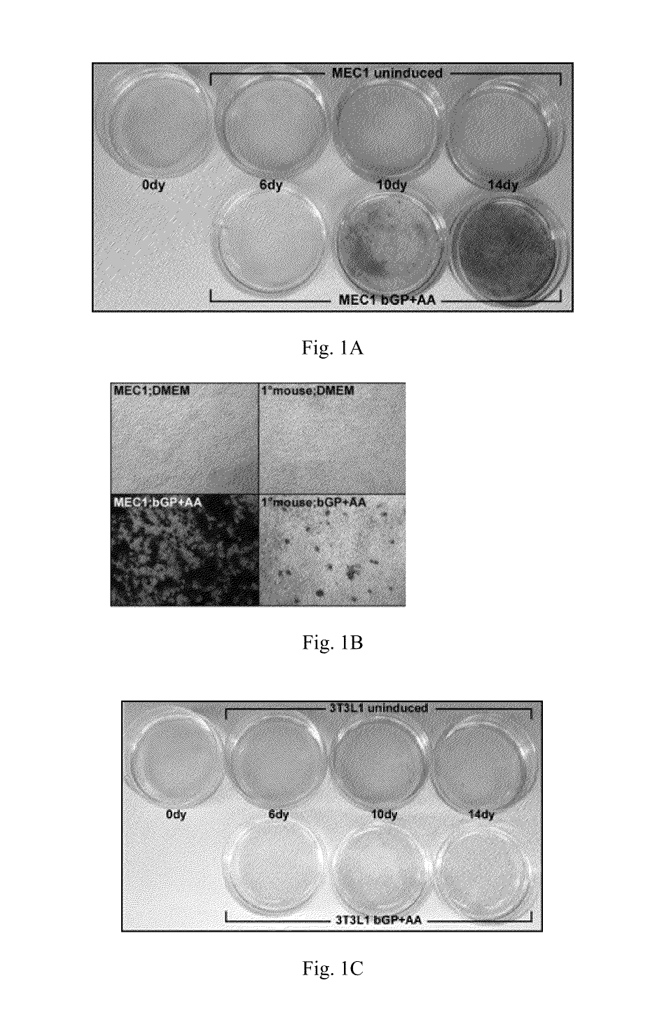

### DETAILED DESCRIPTION

Two established fates of epicardium-derived cells (EPDCs), referred to herein as “typical fates” of epicardium-derived cells are to become smooth muscle cells or fibroblasts [1], and there is evidence that the choice of fibroblast vs. smooth muscle fate is temporally regulated and occurs even when the cells are still within the epicardium itself [2]. In other parts of the embryo, mesenchymal cells give rise to additional cell types, including osteoblasts and adipocytes.

One aspect of the present invention is directed to the ability and/or potential of EPDCs to differentiate to fates that are commonly adopted by mesenchymal cells elsewhere in the body, but not currently associated with the epicardium. Specifically, one aspect of this invention is directed to methods, systems and assays relating to adipocyte and/or osteogenic differentiation that are often related to disease or disregulated states, i.e. the “atypical” fates of epicardium-derived cells—as wells as the “typical fates”. Without being limited to theory, it is believed that both allegedly “atypical” differentiation pathways are in fact followed naturally in the body and in contexts that are clinically relevant, and yet have received little attention in the past. One aspect of the present invention is directed to specific EPDC cells that model both the typical fates and atypical fates of EPDCs, and resultant uses of such EPDC cells in systems, methods and compositions.

Further, in the mouse, chick, and fish (i.e., the primarily experimental systems), the heart does not normally have osteoblasts or adipocytes, and so these fates have been refractory to analysis due to the lack of a suitable model for osteoblast or adipocyte formation in the primary experimental system.

Another aspect of the present invention is the based on the finding that mouse epicardium has the capacity to generate osteoblasts and adipocytes, although it normally doesn't do so in vivo and so mouse EPDCs as described hereincan form the basis for research tools, assays, methods and systems for the elucidation of osteoblast-like and adipocyte fates of epicardial derived cells as well as smooth muscle cells or fibroblasts.

### Preferred Cells and Cell Lines and Methods Based Thereon

The cells and cell lines generally useable in connection with the methods, systems and assays of the present invention generally include primary mammal embryonic epicardial cells, and cells from epicardial derived cell lines, and preferably cells from stable, immortalized cell lines derived from epicardial cells. Additionally, the cells and cell lines generally useable in connection with methods systems and assays of the present invention include (1) primary mammalian epicardial cells, (2) MEC1 cells, (3) modified primary mammalian epicardial cells that are modified to express, overexpress or knockdown expression of at least one of Runx2, PPARγ and/or its upstream transcriptional regulator C/EBPβ and (4) MEC1 cells that are modified to express, overexpress or knock down the expression of at least one of Runx2, PPARγ and/or its upstream transcriptional regulator C/EBPβ. One aspect of the present invention is directed to composition comprising modified primary mammalian epicardial cells and modified MEC1 cell, wherein the cells are modified to express, overexpress or knock down the expression of at least one of Runx2, PPARγ and/or its upstream transcriptional regulator C/EBPβ when compared to relevant control cells that are unmodified.

We previously described methods for the derivation and culture of primary mouse embryonic epicardial cells [11-12]. Similar approaches to culture adult primary epicardial cells from mouse, rabbit, and pig have been accomplished. Such approaches will result in the culturing human primary epicardial cells.

In addition, in Li P, Cavallero S, Gu Y, Chen T H, Hughes J, Hassan A B, Bruning J C, Pashmforoush M, Sucov H M (2011, “IGF signaling directs ventricular cardiomyocyte proliferation during embryonic heart development,” Development 138: 1795-1805, which is incorporated herein by reference in its entirety, we characterized and described the isolation of a spontaneously immortalized mouse embryonic ventricular epicardial cell line, which we termed MEC1 [12]. Due to their ease of culture and clonal nature, MEC1 cells are a preferred cell in connection with the assays, methods and systems of the present invention. MEC1 is a stable epicardial cell line from the embryonic ventricle. Starting from primary epicardial cell cultures from E13.5 wild-type ventricles, individual colonies were manually subcloned that retained the morphology of early primary epicardial cells. After several rounds of subcloning, a stable cell line was obtained, referred to herein as MEC1 (mouse epicardial cells). In addition to having an epicardial morphology, MEC1 cells express a number of epicardial-specific markers, including epicardin (Tcf21), Tbx18 and keratin 18, but do not express markers of myocardial or endothelial cell lineages. MEC1 cells are generally immunopositive for nuclear WT1, a marker that is expressed in epicardial epithelium and in epicardial cells that undergo epithelialmesenchymal transformation, but which is downregulated as these cells differentiate. The MEC1 cell line has been passaged without apparent loss of morphology or of marker gene expression and thus appears to be stably immortalized.

When grown to a density where cell-cell contact occurs, MEC1 cells are epithelial in morphology, cell surface protein localization, and gene expression [12]. However, at lower density, these cells express mesenchymal markers such as smooth muscle actin and vimentin (and others described below). Because the MEC1 cell line retains this property with subcloning and passage, it is believed it has plasticity to oscillate between an epithelial and mesenchymal state depending on density.

The primary mammalian epicardial cells and MEC1 may be modified to express, overexpress or knock down the expression of at least one of Runx2, alkaline phosphatase, PPARγ and/or its upstream transcriptional regulator C/EBPβ.

One aspect of the present invention is directed to primary mammalian epicardial cells and MEC1 that are modified to express, overexpress or knock down the expression of at least one of Runx2, PPARγ and/or its upstream transcriptional regulator C/EBPβ. The term “modified to express” means to force or confer expression in a cell that does not detectably express a feature or structure in its native state. Overexpression and knocked down expression means increased or decreased express compared to unmodified cells of the same type. Lentiviruses may be used that drive Runx2 overexpression (the “bone-type” isoform identified herein) or that knock down endogenous Runx2 expression (all isoforms as identified herein) via an shRNA. In both cases, expression can be controlled by a doxycycline-inducible promoter, which allows very precise manipulation of expression levels, and there is no ectopic expression in cells after infection without doxycycline. The constructs may also include a hygromycin marker for selection of infected cells (if needed). These lentivirus constructs have been validated [28]. Thus, another aspect of the present invention is directed to a method for controlling osteogenic differentiation comprising contacting to primary mammalian epicardial cells and MEC1 with a construct that modifies Runx2 expression in the cell.

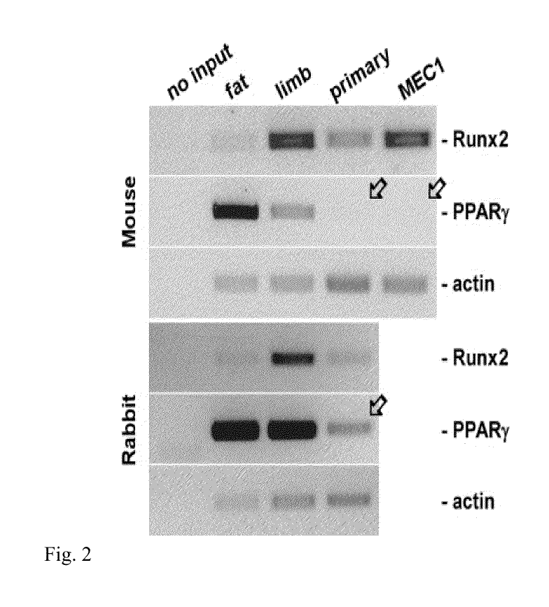

Runx2 is at the top of a hierarchy of transcriptional control that is required for osteoblast differentiation. Runx2 is expressed basally in both MEC1 cells and in primary mouse EPDCs (FIG. 2). Without being limited to theory, Runx2 expression is related to the propensity of these cells to initiate osteoblast differentiation. Another osteoblast marker, alkaline phosphatase, is also expressed in the epicardium, which can be confirmed by the histochemical detection of alkaline phosphatase activity.

Runx2 is a complex protein with many functions; it promotes osteogenic differentiation, but in addition, Runx2 also promotes mesenchymal transformation (this is particularly well studied in cancer metastasis [13-14]). There are two isoforms of Runx2 generated by differential promoter usage. Mice lacking all Runx2 isoforms survive embryogenesis but completely lack bone and die immediately upon birth [15-17]. In contrast, mice lacking the “bone-specific” Runx2-II isoform survive through adulthood and have bone[18-19], implying sufficient overlapping expression and functional equivalency by the “mesenchymal” Runx2-I isoform. MEC1 and primary epicardial cells express both isoforms (not shown); we have not yet defined which is in higher abundance. Without being limited to theory, it is proposed that that Runx2 expression in mouse EPDCs is a reflection of the mesenchymal nature of these cells, but is also a genetic signature that predisposes mouse EPDCs toward osteogenic differentiation.

BMP2 is a bone-inducing factor and does so at least in part by inducing Runx2 [14,20]. MEC1 cells do not express BMP2, nor did BMP2 treatment induce osteogenic differentiation in MEC1 cell cultures (not shown).

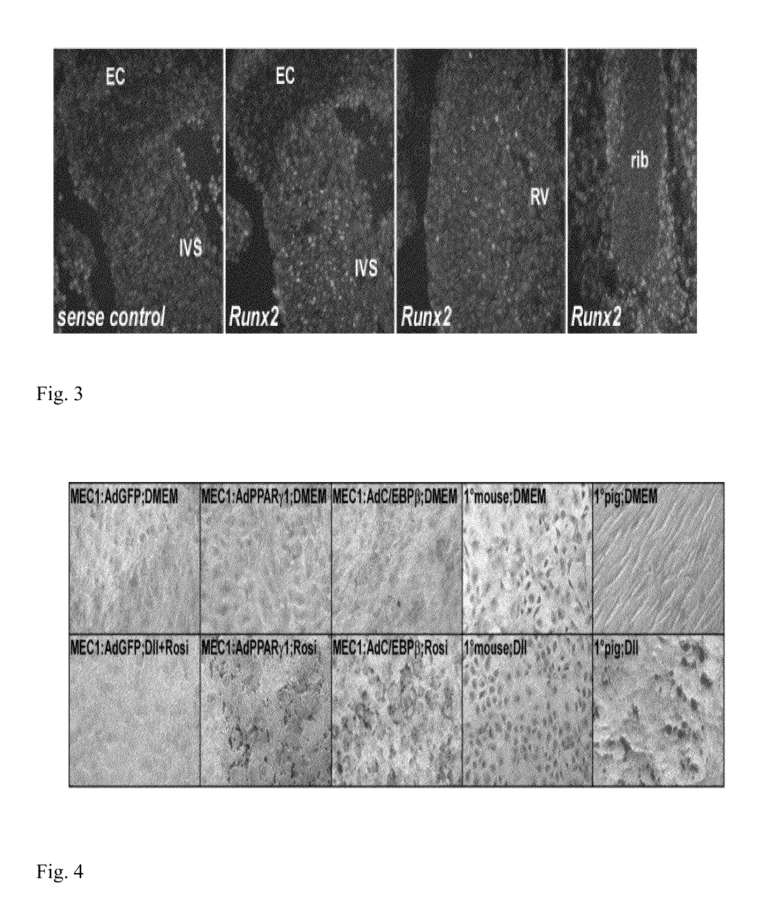

Runx2 expression is also visualized in embryonic heart sections by in situ hybridization (FIG. 3). The epicardium itself was lightly positive although some epicardial cells had more obvious signal. A substantial number of individual cells distributed throughout the ventricular wall and ventricular septum were strongly positive. These cells have the appearance and distribution of cardiac mesenchymal cells seen in other studies [2]. In addition, mesenchymal cells surrounding forming bones were clearly positive, as expected. This Runx2 expression pattern is similar to what is already documented (at lower resolution) in publically available ISH repositories (e.g., [21]).

Although Mouse EPDCs easily differentiate to osteoblast, mouse EPDCs are blocked in adipogenic differentiation, expression of PPARγ or of its upstream transcriptional regulator C/EBPβ in MEC1 cells is sufficient to enable adipogenesis (FIG. 4). A well-studied chemical treatment to induce adipogenic differentiation in cultured preadipocytes is the combination of dexamethasone, insulin, and isobutylmethylxanthine (DII). Using oil red O lipid staining, we observed highly efficient adipocyte differentiation in the preadipocyte cell lines OP9 and NIH3T3L1 (not shown), but MEC1 or primary mouse epicardial cells treated with DII had no positive cells (FIG. 4). This behavior correlates with the lack of cardiac fat in mouse hearts.

At the molecular level, PPARγ is at the top of a hierarchy that leads to adipocyte differentiation. OP9 and NIH3T3L1 cells express PPARγ, and PPARγ expression is necessary for these cell lines to undergo adipogenic differentiation [22]. We observed a striking lack of PPARγ expression in mouse epicardial cells (FIG. 4). PPARγ is a member of the ligand-regulated nuclear receptor family. It has two RNA and thereby protein isoforms generated by alternate promoter usage; the PPARγ2 isoform is highly adipogenic even without ligand, whereas the PPARγ1 isoform is less active basally and requires ligand for full activity.

One aspect of the present invention is directed to compositions comprising the cells of present invention modified in order to express at least one PPARγ, preferably at least one of the PPARγ2 and PPARγ1. An adenovirus may be used to force expression of PPARγ1 [23] in MEC1 cells. In these cells and in response to the synthetic PPARγ ligand rosiglitazone, we observed conversion to adipocytes (FIG. 4). The frequency of conversion is unclear, as we do not yet know the efficiency of infection and the stability of the viral genome in infected cells over time.

Thus, another aspect of the present invention is a method conferring the ability an EPDC to convert to adipocytes comprising contacting the EPDC cell with a construct that forces expression of PPARγ in the EPDC cell.

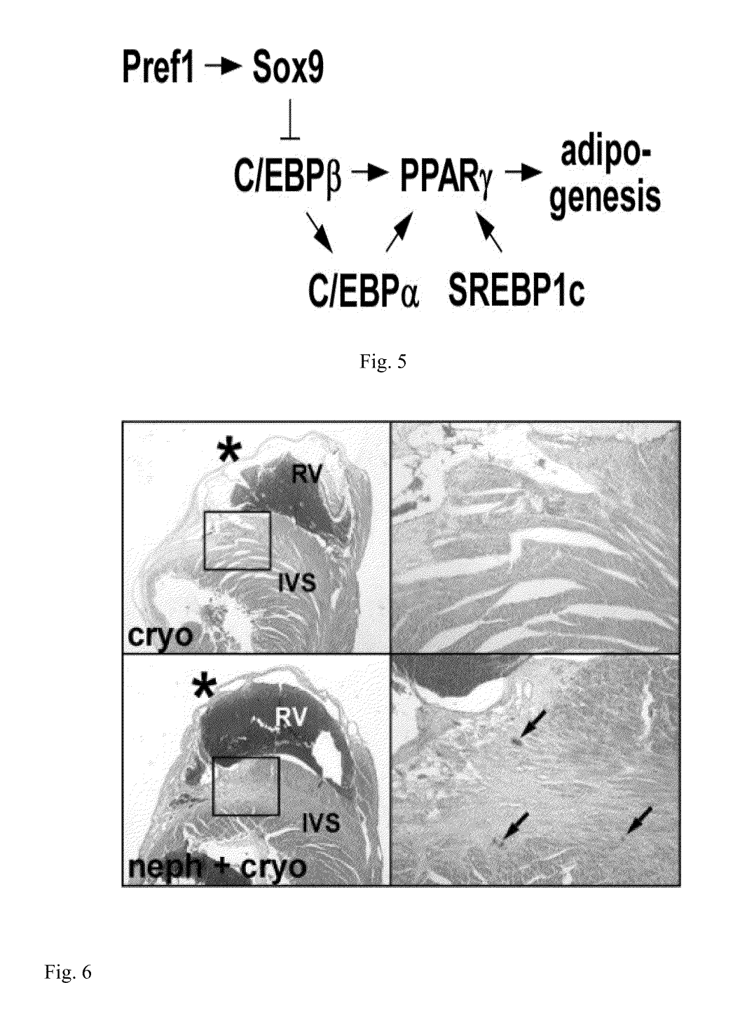

While at the top of the hierarchy of adipogenic differentiation, PPARγ is itself the downstream target of a number of transcription factors (FIG. 5). Adipogenic induction by DII treatment is thought to target different elements of these upstream pathways [24-25]. C/EBPβ is a direct transcriptional regulator of PPARγ expression [26].

Another aspect of the present invention is direct to the epicardial derived cells of the present invention modified to express C/EBPβ. We virally expressed C/EBPβ in MEC1 cells, and observed induction of PPARγ expression (not shown) as well as adipogenic differentiation after rosiglitazone addition (FIG. 4). This demonstrates that the PPARγ gene is not epigenetically silenced in mouse cells, at least in a way that C/EBPβ cannot overcome. Thus, the absence of PPARγ expression and the inability to undergo adipogenic differentiation by mouse EPDCs is a reflection of the activity status of transcriptional circuits upstream of PPARγ.

Another aspect of the present invention is direct to phe primary mammalian epicardial cells and MEC1 that are modified to express, overexpress or knock down the expression of Runx2at least one of Runx2, and that are further modified to express, overexpress or knock down the expression of PPARγ and/or its upstream transcriptional regulator C/EBPβ.

In connection with the compositions, methods and assays of the present invention, the primary epicardial cells, the MEC1 cells, the modified epicardial cells and modified MEC1 cells, and any cells used in connection with the foregoing assays are preferably isolated. The term “isolated” refers to a population that is found in a condition apart from its native environment and apart from other constituents in its native environment, such as blood and animal tissue. The references described herein provide guidance on the purity and isolation of suitable cell populations used and useable in connection with the present invention, and the cell samples described herein are preferably are of a purity and composition consistent with the disclosure of the relevant references.

### Compositions, Assays and Method for Modeling and Drug Discovery for Coronary Artery Calcification

Coronary calcification is characterized by the presence of mineralized calcium in the walls of atherosclerotic coronary vessels. Not all atherosclerotic plaque is calcified, but the presence of calcification is recognized as a major risk factor for plaque rupture [3-5], which can be followed by rapid vessel occlusion and myocardial infarction. Osteoblast-like cells are responsible for mineral deposition in coronary calcification. For lack of a suitable experimental model system, the molecular mechanisms that lead to coronary calcification, and therefore that might be subject to therapeutic intervention, are unknown. One aspect of the present invention is that the mouse epicardium-derived cells useable in connection with the present invention, including MEC1 cells, have osteoblast-like properties.

Another aspect of the present invention is directed to compositions, methods and assays useful for the identification of therapeutic agents that selectively inhibit coronary calcification without inhibiting normal osteoblast formation and associated desirable bone growth.

One aspect of the invention is directed to a composition comprising: EPDC cells of the present invention, preferably MEC1 cells, in a medium, preferably an aqueous medium, and at least one phosphate source. The EPDC cells of the invention are generally at a high enough density in the medium and the phosphate source in the medium is present at concentrations sufficient to produce a calcium mineralized material dispersed or deposited in an extracellular matrix, and preferably a calcium phosphate mineralized material dispersed or deposited in the extracellular matrix. The calcium source is not particularly limited and may generally be any calcium source known to those ordinarily skilled to be useable in connection with the deposition of calcium mineralization from osteoblasts. In an especially preferred embodiment, the calcium source is beta-glycerolphosphate. Preferably, the medium further includes ascorbic acid, and preferably the medium includes both beta-glycerolphosphate and ascorbic acid (bGP+AA).

A method of identifying a compound for preventing and/or treating coronary calcification or a disease state associated with coronary calcification comprises (1) obtaining a EPDC cell, preferably an MEC1 cell, and providing the MEC1 cell(s) in a medium comprising a phosphate source, (2) adding a test compound to the medium, in the presence of the EPDC cells and the phosphate source, (3) measuring a calcium mineralization response, (4) comparing the mineralization response in the sample comprising the EPDC cells, phosphate source and test compound with a calcium mineralization response of in a control without the test compound, and (5) selecting a test compound that reduces the mineralization response in the presence of test compound compared to a mineralization response in the control. If the test compound is found to have reduced the calcium mineralization compared to calcium mineralization in a medium having EPDC cells in which the test compound is not administered, the test compound can be identified as a compound for preventing and/or treating coronary calcification or a disease state associated with coronary calcification. Although the method has been described in terms of adding a test compound to a medium comprising EPDC and the phosphate source, the methods of the present invention may also be practice by adding EPDC and/or aa phosphate source to a medium comprising a test compound as would be understood by those ordinarily skilled.

The measured calcium mineralization response is preferably, but is not limited to, the deposition of calcium mineralized material deposited in an extracellular matrix. In such embodiments, the EPDC cells of the invention should generally be at a high enough density in the medium and the phosphate source in the medium should present at a concentration sufficient to produce a calcium mineralized material dispersed or deposited in an extracellular matrix, and preferably a calcium phosphate mineralized material dispersed or deposited in the extracellular matrix. The calcium mineralized material may be detected and quantified by any suitable method known to those ordinarily skill in the art, including visual observation or staining.

The composition used in connection with the methods generally comprise: EPDC cells of the present invention, preferably MEC1 cells, in a medium, preferably an aqueous medium, and at least one phosphate source. The EPDC cells of the invention are generally at a high enough density in the medium and the phosphate source in the medium is present at concentrations sufficient to produce a calcium mineralized material dispersed or deposited in an extracellular matrix, and preferably a calcium phosphate mineralized material dispersed or deposited in the extracellular matrix. The calcium source is not particularly limited and may generally be any calcium source known to those ordinarily skilled to be useable in connection with the deposition of calcium mineralization from osteoblasts. In an especially preferred embodiment, the calcium source is beta-glycerolphosphate. Preferably, the medium further includes ascorbic acid, and preferably the medium includes both beta-glycerolphosphate and ascorbic acid (bGP+AA).

In a preferred embodiment, the method of the present invention further comprises a method of determining that the test compound selectively inhibits osteoblast-like formation in EPDC without, for instance, inhibiting osteoblast formation resulting from mesenchymal cells elsewhere in the body and desired appropriate bone-formation (a “reference osteoblast”). Preferably, the method further comprises a step of (1) obtaining a reference osteoblast from a non-epicardial source or an reference osteoblast from a bone-producing source, and providing the reference osteoblast in a medium comprising a phosphate source, (2) adding the test compound to the medium, in the presence of the reference osteoblast cells and the phosphate source, (3) measuring a calcium mineralization response in the medium, (4) comparing the mineralization in the medium comprising the reference osteoblast, phosphate source and test compound with a calcium mineralization response of in a control medium without the test compound, and (5) selecting a test compound that produces a mineralization response in the presence of test compound that is substantially the same as the mineralization response in the control. If the test compound is found to have substantially the same calcium mineralization compared to calcium mineralization in a medium having the reference osteoblast cells in which the test compound is not administered, the test compound can be identified as a compound for selectively preventing and/or treating coronary calcification or a disease state associated with coronary calcification.

Although the method has been described in terms of adding a test compound to a medium comprising a reference osteoblast and the phosphate source, the methods of the present invention may also be practice by adding the reference and/or a phosphate source to a medium comprising a test compound as would be understood by those ordinarily skilled.

These compositions and methods can be used as assays for the identification of compounds for use in preventing and or treating coronary calcification or a disease state associated with coronary calcification. These compositions and methods can be used under suitable assay conditions. As used herein, the term “suitable assay conditions” is intended to mean conditions under which a particular assay will identify a compound. Suitable assay conditions take into account factors such as the concentration of the compound, the duration of contact with the compound, the temperature and buffer conditions, the method of contact, whether or not cell viability is required, and the detection format. Suitable assay conditions can depend on the number of compounds being screened. Assay conditions to identify compounds that alter predetermined properties of cells are known in the art or can be readily determined for a particular application of the method.

The assays and associated methods and compositions are based in part on the observation that epicardial cells useable in connection with the present invention can be induced to undergo osteoblast differentiation by treatment with beta-glycerolphosphate and ascorbic acid (bGP+AA). Osteoblast differentiation is commonly evaluated by the presence of mineralized matrix as visualized by alizarin red S staining Untreated MEC1 cell cultures remained undifferentiated even when cultures reached excessively high density, but there was massive deposition of alizarin-stained material when MEC1 cells were treated with bGP+AA (FIGS. 2 and 3). The osteoblast differentiation genes AP, osterix, and BSP were induced by treatment (not shown). The outcome with primary mouse EPDCs (both embryonic and adult) was similar (FIG. 3); the extent of mineralization in induced primary cell cultures was less than with MEC1 cells, which might reflect the lesser density of cells (overt mineralization requires cells to aggregate into nodules, which is density dependent) or some heterogeneity in cellular phenotype. Despite the absence of mineralized matrix in the normal embryonic and postnatal heart, the conclusion from this analysis is that MEC1 cells and at least a prominent subset of mouse primary EPDCs are able to undergo osteoblast differentiation.

As indicated previously herein, the EPDC of the present invention have the capacity to generate osteoblasts and adipocytes as well as smooth muscle cells or fibroblasts. As would be readily apparent to those ordinarily skilled, the methods and assays of the present application can be used for the identification of therapeutic agents that selectively inhibit or enhance at least one of osteoblast formation, adipocyte formation, smooth muscle cell formation or fibroblast formation in EPDC cells. Such assays generally comprise (1) obtaining an EPDC cell, preferably an MEC1 cell, and providing the MEC1 cell(s) in a medium under conditions that result in at least one of osteoblast formation, adipocyte formation, smooth muscle cell formation or fibroblast formation in EPDC cells, (2) adding a test compound to the medium, in the presence of the EPDC cells under the conditions that result in the selected one of osteoblast formation, adipocyte formation, smooth muscle cell formation or fibroblast formation, (3) measuring an amount the selected one of osteoblast formation, adipocyte formation, smooth muscle cell formation or fibroblast formation or a surrogate marker of the selected one in the medium, (4) comparing the selected one of osteoblast formation, adipocyte formation, smooth muscle cell formation or fibroblast formation in the presence of the test compound with a suitable control, and (5) selecting a test compound that reduces or enhances the selected one of osteoblast formation, adipocyte formation, smooth muscle cell formation or fibroblast formation as compared to the control. If the test compound is found to have reduced or enhanced the selected one of osteoblast formation, adipocyte formation, smooth muscle cell formation or fibroblast formation relative to the control, the test compound can be identified as a compound for preventing, enhancing and/or treating the selected one of osteoblast formation, adipocyte formation, smooth muscle cell formation or fibroblast formation and disease states associated therewith.

Selectivity of the compound may be assessed by providing the desired reference cell whose function should be substantially unchanged and by measuring the activity of the reference cell in the presence of the test compound relative to a control in which no test compound is present.

### Animal Model Coronary

After the embryonic period of EMT, the epicardium becomes quiescent. However, adult heart injury reactivates the epicardium and reinitiates EMT [37]. Mice do not generally develop coronary calcification, even under conditions and in genetic backgrounds where abundant calcification occurs elsewhere (e.g., [40-42])

Thus, another aspect of the present invention is a method for inducing coronary artery calcification in non-human mammals, preferably mice—one that could be adapted for testing of therapeutics. The method comprises 5/6 nephrectomy in the adult non-human mammal and then later followed with cryoinjury to the heart of the mammal.

Another aspect of the present invention is directed to an animal model comprising a non-human mammal having induced coronary artery calcification. The non-human mammal is preferably a mouse.

One advantage of the present invention does not require genetic mutations nor drug/diet treatments. The method of inducing coronary artery calcification of the present invention is designed to mimic human renal failure patients, who have a much higher incidence of coronary calcification, and for which there may not be any other experimental model.

In humans, kidney disease (renal failure) is one of the most significant risk factors for coronary artery calcification, such that heart attack is a leading cause of death among renal failure patients [43-44]. Kidney disease is associated with elevated serum phosphate levels [45], which is thought to activate osteogenesis in vivo much as bGP does in cell culture [43]. In rodents, 5/6 nephrectomy (one kidney removed in full, the other resected to leave only the central one-third) is a model of renal failure, but is not associated with coronary calcification. Human kidney failure patients generally have other cardiovascular issues, including coronary atherosclerosis and infarction, which are not seen in nephrectomized mice. Atherosclerosis and infarction are associated with inflammation, and inflammation is highly associated with the onset of calcification [46-47]. Thus, we speculated that renal failure combined with an inflammatory environment might synergistically induce coronary artery calcification in a way that would allow us to study the role of EPDC osteogenic differentiation.

To model this in mice, we 5/6 nephrectomized adult mice and then later followed with cryoinjury to the heart. The recovery from cryoinjury is associated with a strong inflammatory response [48-49]. In the vicinity of the injury zone and to a lesser extent throughout the rest of the ventricle, we clearly observed alizarin staining (mineralized calcium) within small caliber vessels (FIG. 6). Von Kossa staining (mineralized phosphate) showed a similar pattern (not shown). Importantly, this staining was only seen with the combination of nephrectomy and injury; cryoinjury alone (FIG. 6) or nephrectomy alone (not shown) did not result in any staining whatsoever. We are undertaking a more detailed characterization of this model; it will be worthwhile for example to measure serum phosphate to confirm that this is the priming condition that facilitates subsequent osteogenesis. Nonetheless, this is an experimental model amenable to systematic analysis that will allow us to test the hypothesis that Runx2 expression confers osteogenic potential to EPDCs in vivo in the adult heart as it does in cell culture.

### REFERENCES

All references cited herein, including the foregoing, are incorporated herein by reference in their entirety.
1. von Gise A, Pu W T (2012) Endocardial and epicardial epithelial to mesenchymal transitions in heart development and disease. Circ Res 110: 1628-1645.
2. Acharya A, Baek S T, Huang G, Eskiocak B, Goetsch S, Sung C Y, Banfi S, Sauer M F, Olsen G S, Duffield J S, Olson E N, Tallquist M D (2012) The bHLH transcription factor Tcf21 is required for lineage-specific EMT of cardiac fibroblast progenitors. Development 139: 2139-2149.
3. Sharma R K, Voelker D J, Singh V N, Pahuja D, Nash T, Reddy H K (2010) Cardiac risk stratification: role of the coronary calcium score. Vasc Health Risk Manag 6: 603-611.
4. Vliegenthart R, Oudkerk M, Hofman A, Oei H H, van Dijck W, van Rooij F J, Witteman J C (2005) Coronary calcification improves cardiovascular risk prediction in the elderly. Circulation 112: 572-577.
5. Schenker M P, Dorbala S, Hong E C, Rybicki F J, Hachamovitch R, Kwong R Y, Di Carli M F (2008) Interrelation of coronary calcification, myocardial ischemia, and outcomes in patients with intermediate likelihood of coronary artery disease: a combined positron emission tomography/computed tomography study. Circulation 117: 1693-1700.
6. Bedford E (1972) The story of fatty heart. A disease of Victorian times. Br Heart J 34: 23-28.
7. Marchington J M, Mattacks C A, Pond C M (1989) Adipose tissue in the mammalian heart and pericardium: structure, foetal development and biochemical properties. Comp Biochem Physiol B 94: 225-232.
8. Fletcher B D, Jacobstein M D, Abramowsky C R, Anderson R H (1987) Right atrioventricular valve atresia: anatomic evaluation with M R imaging. AJR Am J Roentgenol 148: 671-674.
9. Jornet A, Reig J, Ruiz C, Uson M, Petit M (1990) The intervenous tubercle (of lower): morphological characteristics and changes in relation to age. Arch Anat Histol Embryol 73: 21-32.
10. Schoonderwoerd M, Doige C E, Wobeser G A, Naylor J M (1986) Protein energy malnutrition and fat mobilization in neonatal calves. Can Vet J 27: 365-371.
11. Chen T H, Chang T C, Kang J O, Choudhary B, Makita T, Tran C M, Burch J B, Eid H, Sucov H M (2002) Epicardial induction of fetal cardiomyocyte proliferation via a retinoic acid-inducible trophic factor. Dev Biol 250: 198-207.
12. Li P, Cavallero S, Gu Y, Chen T H, Hughes J, Hassan A B, Bruning J C, Pashmforoush M, Sucov H M (2011) IGF signaling directs ventricular cardiomyocyte proliferation during embryonic heart development. Development 138: 1795-1805.
13. Niu D F, Kondo T, Nakazawa T, Oishi N, Kawasaki T, Mochizuki K, Yamane T, Katoh R (2012) Transcription factor Runx2 is a regulator of epithelial-mesenchymal transition and invasion in thyroid carcinomas. Lab Invest 92: 1181-1190.
14. Hsu Y L, Huang M S, Yang C J, Hung J Y, Wu L Y, Kuo P L (2011) Lung tumor-associated osteoblast-derived bone morphogenetic protein-2 increased epithelial-to-mesenchymal transition of cancer by Runx2/Snail signaling pathway. J Biol Chem 286: 37335-37346.
15. Otto F, Thornell A P, Crompton T, Denzel A, Gilmour K C, Rosewell I R, Stamp G W, Beddington R S, Mundlos S, Olsen B R, Selby P B, Owen M J (1997) Cbfa1, a candidate gene for cleidocranial dysplasia syndrome, is essential for osteoblast differentiation and bone development. Cell 89: 765-771.
16. Komori T, Yagi H, Nomura S, Yamaguchi A, Sasaki K, Deguchi K, Shimizu Y, Bronson R T, Gao Y H, Inada M, Sato M, Okamoto R, Kitamura Y, Yoshiki S, Kishimoto T (1997) Targeted disruption of Cbfa1 results in a complete lack of bone formation owing to maturational arrest of osteoblasts. Cell 89: 755-764.
17. Ducy P, Zhang R, Geoffroy V, Ridall A L, Karsenty G (1997) Osf2/Cbfa1: a transcriptional activator of osteoblast differentiation. Cell 89: 747-754.
18. Liu J C, Lengner C J, Gaur T, Lou Y, Hussain S, Jones M D, Borodic B, Colby J L, Steinman H A, van Wijnen A J, Stein J L, Jones S N, Stein G S, Lian J B (2011) Runx2 protein expression utilizes the Runx2 P1 promoter to establish osteoprogenitor cell number for normal bone formation. J Biol Chem 286: 30057-30070.
19. Xiao Z S, Hjelmeland A B, Quarles L D (2004) Selective deficiency of the “bone-related” Runx2-II unexpectedly preserves osteoblast-mediated skeletogenesis. J Biol Chem 279: 20307-20313.
20. Banerjee C, Javed A, Choi J Y, Green J, Rosen V, van Wijnen A J, Stein J L, Lian J B, Stein G S (2001) Differential regulation of the two principal Runx2/Cbfa1 n-terminal isoforms in response to bone morphogenetic protein-2 during development of the osteoblast phenotype. Endocrinology 142: 4026-4039.
21. Eurexpress.org. See for example: http://www.eurexpress.org/ee/databases/assay.jsp?assayID=euxassay_{—}019459&image=01.
22. Wolins N E, Quaynor B K, Skinner J R, Tzekov A, Park C, Choi K, Bickel P E (2006) OP9 mouse stromal cells rapidly differentiate into adipocytes: characterization of a useful new model of adipogenesis. J Lipid Res 47: 450-460.
23. Hazra S, Xiong S, Wang J, Rippe R A, Krishna V, Chatterjee K, Tsukamoto H (2004) Peroxisome proliferator-activated receptor gamma induces a phenotypic switch from activated to quiescent hepatic stellate cells. J Biol Chem 279: 11392-11401.
24. Farmer S R (2006) Transcriptional control of adipocyte formation. Cell Metab 4: 263-273.
25. Wang Y, Sul H S (2009) Pref-1 regulates mesenchymal cell commitment and differentiation through Sox9. Cell Metab 9: 287-302.
26. Tang Q Q, Zhang J W, Lane M D (2004) Sequential gene promoter interactions by C/EBPbeta, C/EBPalpha, and PPARgamma during adipogenesis. Biochem Biophys Res Commun 318: 213-218.
27. Jefcoate C R, Wang S, Liu X (2008) Methods that resolve different contributions of clonal expansion to adipogenesis in 3T3-L1 and C3H10T1/2 cells. Methods Mol Biol 456: 173-193.
28. Baniwal S K, Little G H, Chimge N O, Frenkel B (2012) Runx2 controls a feed-forward loop between androgen and prolactin-induced protein (PIP) in stimulating T47D cell proliferation. J Cell Physiol 227: 2276-2282.
29. Okuda T, van Deursen J, Hiebert S W, Grosveld G, Downing J R (1996) AML1, the target of multiple chromosomal translocations in human leukemia, is essential for normal fetal liver hematopoiesis. Cell 84: 321-330.
30. Wang Q, Stacy T, Binder M, Marin-Padilla M, Sharpe A H, Speck N A (1996) Disruption of the Cbfa2 gene causes necrosis and hemorrhaging in the central nervous system and blocks definitive hematopoiesis. Proc Natl Acad Sci USA 93: 3444-3449.
31. Rauch A, Seitz S, Baschant U, Schilling A F, Illing A, Stride B, Kirilov M, Mandic V, Takacz A, Schmidt-Ullrich R, Ostermay S, Schinke T, Spanbroek R, Zaiss M M, Angel P E, et al. (2010) Glucocorticoids suppress bone formation by attenuating osteoblast differentiation via the monomeric glucocorticoid receptor. Cell Metab 11: 517-531.
32. Zamora M, Manner J, Ruiz-Lozano P (2007) Epicardium-derived progenitor cells require beta-catenin for coronary artery formation. Proc Natl Acad Sci USA 104: 18109-18114.
33. von Gise A, Zhou B, Honor L B, Ma Q, Petryk A, Pu W T (2011) W T1 regulates epicardial epithelial to mesenchymal transition through beta-catenin and retinoic acid signaling pathways. Dev Biol 356: 421-431.
34. Gaur T, Lengner C J, Hovhannisyan H, Bhat R A, Bodine P V, Komm B S, Javed A, van Wijnen A J, Stein J L, Stein G S, Lian J B (2005) Canonical WNT signaling promotes osteogenesis by directly stimulating Runx2 gene expression. J Biol Chem 280: 33132-33140.
35. Dong Y F, Soung D Y, Schwarz E M, O'Keefe R J, Drissi H (2006) Wnt induction of chondrocyte hypertrophy through the Runx2 transcription factor. J Cell Physiol 208: 77-86.
36. Duan J, Gherghe C, Liu D, Hamlett E, Srikantha L, Rodgers L, Regan J N, Rojas M, Willis M, Leask A, Majesky M, Deb A (2012) Wnt1/betacatenin injury response activates the epicardium and cardiac fibroblasts to promote cardiac repair. EMBO J 31: 429-442.
37. Zhou B, Pu W T (2011) Epicardial epithelial-to-mesenchymal transition in injured heart. J Cell Mol Med 15: 2781-2783.
38. Chen H, Ghori-Javed F Y, Rashid H, Serra R, Gutierrez S E, Javed A (2011) Chondrocyte-specific regulatory activity of Runx2 is essential for survival and skeletal development. Cells Tissues Organs 194: 161-165.
39. Sun Y, Byon C H, Yuan K, Chen J, Mao X, Heath J M, Javed A, Zhang K, Anderson P G, Chen Y (2012) Smooth muscle cell-specific runx2 deficiency inhibits vascular calcification. Circ Res 111: 543-552.
40. Yao Y, Bennett B J, Wang X, Rosenfeld M E, Giachelli C, Lusis A J, Bostrom K I (2010) Inhibition of bone morphogenetic proteins protects against atherosclerosis and vascular calcification. Circ Res 107: 485-494.
41. Schurgers L J, Joosen I A, Laufer E M, Chatrou M L, Herfs M, Winkens M H, Westenfeld R, Veulemans V, Krueger T, Shanahan C M, Jahnen-Dechent W, Biessen E, Narula J, Vermeer C, Hofstra L, et al. (2012) Vitamin k-antagonists accelerate atherosclerotic calcification and induce a vulnerable plaque phenotype. PLoS One 7: e43229.
42. Phan O, Ivanovski O, Nikolov I G, Joki N, Maizel J, Louvet L, Chasseraud M, Nguyen-Khoa T, Lacour B, Drueke T B, Massy Z A (2008) Effect of oral calcium carbonate on aortic calcification in apolipoprotein E-deficient (apoE−/−) mice with chronic renal failure. Nephrol Dial Transplant 23: 82-90.
43. Jono S, Shioi A, Ikari Y, Nishizawa Y (2006) Vascular calcification in chronic kidney disease. J Bone Miner Metab 24: 176-181.
44. McCullough P A, Agarwal M, Agrawal V (2009) Review article: Risks of coronary artery calcification in chronic kidney disease: do the same rules apply? Nephrology (Carlton) 14: 428-436.
45. Gutierrez O M (2011) Increased serum phosphate and adverse clinical outcomes: unraveling mechanisms of disease. Curr Opin Nephrol Hypertens 20: 224-228.
46. Li J J, Zhu C G, Yu B, Liu Y X, Yu M Y (2007) The role of inflammation in coronary artery calcification. Ageing Res Rev 6: 263-270.
47. Shao J S, Cheng S L, Sadhu J, Towler D A (2010) Inflammation and the osteogenic regulation of vascular calcification: a review and perspective. Hypertension 55: 579-592.
48. Jensen J A, Kosek J C, Hunt T K, Goodson W H, 3rd, Miller D C (1987) Cardiac cryolesions as an experimental model of myocardial wound healing. Ann Surg 206: 798-803.
49. van Amerongen M J, Harmsen M C, Petersen A H, Popa E R, van Luyn M J (2008) Cryoinjury: a model of myocardial regeneration. Cardiovasc Pathol 17: 23-31.
50. Nuttall M E, Gimble J M (2004) Controlling the balance between osteoblastogenesis and adipogenesis and the consequent therapeutic implications. Curr Opin Pharmacol 4: 290-294.
51. Muruganandan S, Roman A A, Sinal C J (2009) Adipocyte differentiation of bone marrow-derived mesenchymal stem cells: cross talk with the osteoblastogenic program. Cell Mol Life Sci 66: 236-253.
52. Chan L Y, Yeung J H, Lau T K (2005) Placental transfer of rosiglitazone in the first trimester of human pregnancy. Feral Steril 83: 955-958.
53. Klinkner D B, Lim H J, Strawn E Y, Jr., Oldham K T, Sander T L (2006) An in vivo murine model of rosiglitazone use in pregnancy. Feral Steril 86: 1074-1079.
54. Rossini A, Frati C, Lagrasta C, Graiani G, Scopece A, Cavalli S, Musso E, Baccarin M, Di Segni M, Fagnoni F, Germani A, Quaini E, Mayr M, Xu Q, Barbuti A, et al. (2011) Human cardiac and bone marrow stromal cells exhibit distinctive properties related to their origin. Cardiovasc Res 89: 650-660.
55. Smith N, Dong Y, Lian J B, Pratap J, Kingsley P D, van Wijnen A J, Stein J L, Schwarz E M, O'Keefe R J, Stein G S, Drissi M H (2005) Overlapping expression of Runx1(Cbfa2) and Runx2(Cbfa1) transcription factors supports cooperative induction of skeletal development. J Cell Physiol 203: 133-143.
56. Dobson M E, Diallo-Krou E, Grachtchouk V, Yu J, Colby L A, Wilkinson J E, Giordano T J, Koenig R J (2011) Pioglitazone induces a proadipogenic antitumor response in mice with PAX8-PPARgamma fusion protein thyroid carcinoma. Endocrinology 152: 4455-4465.
57. Harty M, Neff A W, King M W, Mescher A L (2003) Regeneration or scarring: an immunologic perspective. Dev Dyn 226: 268-279.
58. Klapka N, Hermanns S, Straten G, Masanneck C, Duis S, Hamers F P, Muller D, Zuschratter W, Muller H W (2005) Suppression of fibrous scarring in spinal cord injury of rat promotes long-distance regeneration of corticospinal tract axons, rescue of primary motoneurons in somatosensory cortex and significant functional recovery. Eur J Neurosci 22: 3047-3058.
59. Chablais F, Jazwinska A (2012) The regenerative capacity of the zebrafish heart is dependent on TGFbeta signaling. Development 139: 1921-1930.

1. A method of identifying a compound for preventing and/or treating coronary calcification or a disease state associated with coronary calcification comprising:
obtaining EPDC cells and providing the EPDC cells in a medium comprising a phosphate source;
adding a test compound to the medium, in the presence of the EPDC cells and the phosphate source;
measuring a calcium mineralization response;
comparing the calcium mineralization response in the sample comprising the EPDC cells, phosphate source and test compound with a calcium mineralization response of in a control without the test compound; and
selecting a test compound that reduces the mineralization response in the presence of test compound compared to a mineralization response in the control.

2. The method of claim 1, wherein the EPDC cells are MEC1 cells.

3. The method of claim 1, wherein calcium mineralization response is the deposition of calcium mineralized in an extracellular matrix of the medium.

4. The method of claim 3, wherein the calcium mineralization response is measured by one of visual observation or staining.

5. The method of claim 1, wherein the medium is an aqueous medium

6. The method of claim 1, wherein the calcium source is beta-glycerolphosphate.

7. The method of claim 6, wherein the medium further comprises ascorbic acid.

8. The method of claim 1, further comprising:
obtaining a reference osteoblast from a non-epicardial source or a reference osteoblast from a bone-producing source;
providing the reference osteoblast in a medium comprising a phosphate source;
adding the test compound to the medium, in the presence of the reference osteoblast cells and the phosphate source;
measuring a calcium mineralization response in the medium;
comparing the mineralization in the medium comprising the reference osteoblast, phosphate source and test compound with a calcium mineralization response of in a control medium without the test compound; and
selecting a test compound that produces a mineralization response in the presence of test compound that is substantially the same as the mineralization response in the control.

9. The composition comprising:
isolated primary epicardial derived cells and a phosphorous source in a aqueous medium, wherein the primary EPDC are at a high enough density in the medium and the phosphate source in the medium is present at sufficient concentrations sufficient to produce a calcium mineralized material dispersed or deposited in an extracellular matrix

10. Epicardial cells selected from the group consisting of primary epicardial cells and MEC1 cells, wherein the epicardial cells express, overexpress or knock down the expression of at least one of Runx2, PPARγ and/or C/EBPβ.

11. The epicardial cells of claim 10, wherein the epicardial cells are MEC1 cells that express PPARγ.

12. The epicardial cells of claim 10, wherein the epicardial cells are MEC1 cells that express C/EBPβ.

13. The epicardial cells of claim 10, wherein the epicardial cells are MEC1 cells that overexpress Runx2.

14. The epicardial cells of claim 10, wherein the epicardial cells are primary epicardial cells that express PPARγ.

15. The epicardial cells of claim 10, wherein the epicardial cells are primary epicardial cells that express C/EBPβ.

16. The epicardial cells of claim 10, wherein the epicardial cells are primary epicardial cells that overexpress Runx2.
